(19) United States
(12) Patent Application Publication
(10) Pub. No.: US 2006/0024340 A1
(43) Pub. Date: Feb. 2, 2006

(54) Encapsulated fluorescent whitening compositions for improved surface appearance

(76) Inventors: Stewart Todd Elder, Butler, NJ (US); Christina Ligia Andrianov, Monroe, NY (US)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 10/903,642

(22) Filed: Jul. 30, 2004

(51) Int. Cl. A61K 8/49

(52) U.S. Cl. 424/401; 424/63

(57) ABSTRACT

Microparticles containing an effective amount of at least one fluorescent whitening agent, wherein said fluorescent whitening agent is entrapped in a transparent or translucent matrix polymer that has been formed from a blend of monomers comprising a first monomer which is an ethylenically unsaturated ionic monomer and a second monomer which is an ethylenically unsaturated hydrophobic monomer which is capable of forming a homopolymer of glass transition temperature in excess of 50° C., wherein secondary particles are distributed throughout the matrix, which secondary particles comprise a hydrophobic polymer that has been formed from an ethylenically unsaturated hydrophobic monomer which is capable of forming a homopolymer having a glass transition temperature in excess of 50° C. and optionally other monomers, which hydrophobic polymer is different from that of the matrix polymer, exhibit improved shatter resistance. They are useful in a personal care or cosmetic compositions, for example in masking or reducing the appearance of skin imperfections.

### FIELD OF THE INVENTION

This invention relates an encapsulated fluorescent whitening agent. More particularly it relates to shatter-resistant microcapsules comprising at least one fluorescent whitening agent and their preparation, a composition comprising shatter-resistant microcapsules of at least one fluorescent whitening agent and use thereof in personal care applications.

### BACKGROUND OF THE INVENTION

There is a need to provide fluorescent particles with improved shatter resistance that can be used for a variety of applications. Specifically there is a need to provide products containing entrapped or encapsulated fluorescent whitening agents (FWAS), which products retain the fluorescent whitening agent over extended periods and exhibit improved shatter resistance when subjected to different environments. This is particularly the case when employing oil soluble and water-soluble fluorescent whitening agents, where it is generally difficult to permanently retain the FWA. In a cosmetic composition if the FWA is not permanently retained, this can impair the long-term visual effect of the cosmetic.

U.S. Pat. No. 5,234,711 describes a method for encapsulation of pigment particles utilized in ink formulations and their use for cosmetic products. The cosmetic products are especially directed to eyeliner pens.

U.S. Pat. No. 5,382,433 and published PCT Application WO 98/5002 describe the use of a cosmetic stick that contains microencapsulated pigment particles. The encapsulated pigment in the '433 patent is made by coacervation polymerization. The PCT application expands on this patent by including a volatile solvent in the cosmetic composition. The volatile solvent is present to minimize the gritty feel of the microencapsulated material.

U.S. Pat. No. 5,234,711 concerns methods of encapsulating pigment particles useful in manufacturing of cosmetic products. It is an objective of this reference to employ an encapsulation process to increase the wettability, dispersibility and heat resistance of the pigment particles. The encapsulation method involves redox or free radical vinyl polymerization in an aqueous medium.

Published European Patent Application 225,799 describes a microencapsulated solid non-magnetic colorant material in a liquid, gel, wax or low temperature melting solid carrier phase, which is encapsulated within a polymeric shell. Absorbed onto the shell is a silane or titanate coupling agent, which increases the oleophilicity of the surface of the solid colorant material.

Published European Patent Application 445,342 relates to a cosmetic composition comprising a pigment that has been formed by incorporating a solvate dye into a resin and admixing with a cosmetic carrier. The amount of pigment present is sufficient to provide an attractive cosmetic effect when applied to skin, nails or hair. Any cosmetically acceptable soluble dye can be used. Any resin may be used provided it can be pulverized to a fine powder. The solvate dye may be incorporated into the resin by adding it to the elasticized or molten resin, or by dissolving the dye in a solution of unpolymerized resin and a mutual solvent for the dye and the resin, then polymerizing the resin, or by contacting the dye with the resin. The dye-impregnated resin powders are said to be usable in a variety of cosmetic compositions.

WO 02/090445 addresses the problem of color retention and provides polymeric particles comprising a matrix polymer and colorant distributed throughout it. The matrix polymer is formed from a blend of monomers comprising a first monomer, which is an ethylenically unsaturated ionic monomer, which is a salt of a volatile counterion, and a second monomer, which is an ethylenically unsaturated hydrophobic monomer, which is capable of forming a homopolymer of glass transition temperature in excess of 50° C. Typical matrix polymers include copolymers that have been formed from styrene with ammonium acrylate. The polymeric particles exhibit good retention properties and are able to retain the colorant under a variety of conditions. However, these particles tend to suffer the drawback that they can fracture and even shatter under certain conditions when handled harshly, and this can lead to loss of the colorant.

U.S. Patent Application Publication No. 2002/0192260 A1 discloses optically activated particles for use in cosmetic preparations to reduce the visual perception of skin imperfections. The optically-activated particles are of various substrates such as nylons, acrylics, polyesters, other plastic polymers, natural materials, regenerated cellulose, metals and minerals, and having an optical brightener chemically bonded to the substrate particles to form integral units in the form of optically-activated particles for diffusing and emitting light to reduce the visual perception of cellulite, shadows, skin discolorations and wrinkles. Each of the optically-activated particles may be additionally encapsulated with a UV transparent coating, for example a polyoxymethylene urea, to increase the diffusion of light to further reduce the visual perception of cellulite, shadows, skin discolorations and wrinkles. According to page 3, paragraph [0029] of the disclosure, the optical brightener compound is chemically bonded to the substrate (e.g. a nylon spheroid particle) by covalent or ionic bonding, such that the optical brightener is inseparable from the nylon particle and becomes part of the finished optically-activated particle. In the present invention the optical brightener (FWA) is not bonded to a particle.

Copending U.S. patent application Ser. No. 10/785,208 describes the use of a blend of microencapsulated colorants prepared as described in WO 02/090445 in cosmetic compositions. The blend produces a textured natural tone coloring when applied, or creates similar effects on or in the cosmetic product itself. However, as noted below, the microcapsules are structurally different from those employed according to the present invention and lack their shatter-resistance.

Encapsulation or entrapment of fluorescent whitening agents can result in visual impairment of them. This may be as a result of the polymer absorbing light from certain wavelengths or sometimes as a result of the irregular morphology of the polymer particles. This is also true where the particles are not shatter resistant. Fractures in the particles or broken particles will also lead to visual impairment of the fluorescent whitening agent.

Various methods for making capsules have been proposed in the literature. For instance it is known to encapsulate hydrophobic liquids by dispersing the hydrophobic liquid into an aqueous medium containing a melamine formaldehyde pre-condensate and then reducing the pH. This results in an impervious aminoplast resin shell wall surrounding the hydrophobic liquid. Variations of this type of process are described in GB-A-2073132, AU-A-27028188 and GB-A-1507739, in which the capsules are preferably used to provide encapsulated inks for use in pressure sensitive carbonless copy paper.

However, although capsules based on melamine formaldehyde resins are both impervious and durable, they tend to suffer the disadvantage that they are less impermeable at elevated temperatures. In addition there is also a risk that at elevated temperatures formaldehyde is evolved.

Typical techniques for forming a polymer shell are described in GB 1,275,712, 1,475,229 and 1,507,739, DE 3,545,803 and U.S. Pat. No. 3,591,090 for instance.

In EP-A-356,240 processes for encapsulating enzyme or other biologically produced material in a matrix of polymeric material by mixing the polymeric material with aqueous liquor containing the biologically produced material, dispersing this mixture in a water immiscible liquid and azeotroping the dispersion are disclosed.

The product can either be relatively coarse beads that can be recovered or a stable dispersion of small particles in the water immiscible liquid. In EP-A-356,239 there is a description of various compositions and processes primarily intended for the encapsulation of enzymes for liquid or other detergents. The particles are designed to subsequently release the enzyme during laundering.

Particles of a matrix polymer containing an active ingredient can be formed as a dispersion in oil, and this dispersion can then be dispersed in aqueous solution of an encapsulating polymer or blend of polymers. Polymer deposition can then be caused to occur around the oil particles that contain the particles of matrix polymer that contain the active ingredient.

U.S. Pat. No. 5,744,152 describes a process for forming polymer particles introduced as a solution of a water soluble salt of a volatile amine of a polymer that is relatively insoluble and non-swelling in acid, throughout which the active ingredient is dispersed or dissolved, and which solution is heated to form a dry matrix and to volatilize the amine, thereby forming a polymer that is insoluble in acid. The release of an active ingredient can be controlled by careful adjustment of the pH.

This method is specifically designed for the entrapment of relatively large sized ingredients, in particular enzymes, fungi, spores, bacteria, cells or antibiotics, which are released by pH adjustment as a suitable release mechanism.

WO 97/24178 describes a particulate composition comprising particles having a polymeric matrix including a detergency active ingredient, wherein the polymeric matrix is formed of a free base form of a cationic polymer which is a co-polymer of an ethylenically unsaturated hydrophobic monomer with an ethylenically unsaturated substituted amine monomer. The matrix particles can be made by polymerizing the free base monomer and the hydrophobic monomer while dissolved in an organic solvent so as to form a solution of the free base polymer in the inorganic solvent, followed by addition of an aqueous solution of a volatile acid that has a lower volatility than the solvent. The solvent is then distilled off so as to leave a solution in water of the salt form of the polymer. A suitable volatile acid is acetic acid, in which event a suitable solvent is n-butyl acetate. The active ingredients particularly include enzymes that can be released by dilution of the medium in which they are contained.

Many of the aforementioned references are concerned with entrapment or encapsulation of active ingredients, which are to be released at a later stage and thus do not give any indication of how to achieve permanent entrapment of materials, particularly relatively small sized species.

U.S. Pat. No. 5,234,711 concerns methods of encapsulating pigment particles useful in manufacturing of cosmetic products. It is an objective of this reference to employ an encapsulation process to increase the wettability, dispersibility and heat resistance of the pigment particles. The encapsulation method involves redox or free radical vinyl polymerization in an aqueous medium.

The aforementioned prior art does not describe the use of microencapsulated fluorescent whitening agents (FWAS) that are not bonded to a particulate substrate in personal care applications. Nor does it describe shatter-resistant microcapsules comprising at least one fluorescent whitening agent

An object of the present invention is to provide a shatter-resistant microcapsule comprising at least one fluorescent whitening agent. Still another is to provide a cosmetic composition comprising shatter-resistant microcapsules of at least one fluorescent whitening agent, whereby the compositions retain the FWA over extended periods and also when subjected to different environments. This is especially important when the FWAS are oil-soluble and particularly so when they are water-soluble, where it is generally difficult to permanently retain them. In a cosmetic composition, if the FWA is not permanently retained, this can impair the visual effect of the cosmetic after prolonged use.

### SUMMARY OF THE INVENTION

In one aspect the present invention provides microparticles containing an effective brightening amount of at least one fluorescent whitening agent, wherein said fluorescent whitening agent is entrapped in a transparent or translucent matrix polymer that has been formed from a blend of monomers comprising a first monomer which is an ethylenically unsaturated ionic monomer and a second monomer which is an ethylenically unsaturated hydrophobic monomer which is capable of forming a homopolymer of glass transition temperature in excess of 50° C., wherein secondary transparent or translucent particles are distributed throughout the matrix, which secondary particles comprise a hydrophobic polymer that has been formed from an ethylenically unsaturated hydrophobic monomer which is capable of forming a homopolymer having a glass transition temperature in excess of 50° C. and optionally other monomers, which hydrophobic polymer is different from that of the matrix polymer. The individual fluorescent whitening agent microparticles have a typical particle size of between 1 and 60 microns.

In another aspect the present invention provides a solid or liquid cosmetic composition that comprises a shatter-resistant microparticle containing an effective amount of at least one fluorescent whitening agent, wherein said fluorescent whitening agent is entrapped in one or more transparent or translucent microparticulate matrix polymers that have been formed from a blend of monomers comprising a first monomer which is an ethylenically unsaturated ionic monomer and a second monomer which is an ethylenically unsaturated hydrophobic monomer which is capable of forming a homopolymer of glass transition temperature in excess of 50° C., wherein transparent or translucent secondary particles are distributed throughout the matrix, which secondary particles comprise a hydrophobic polymer that has been formed from an ethylenically unsaturated hydrophobic monomer which is capable of forming a homopolymer having a glass transition temperature in excess of 50° C. and optionally other monomers, which hydrophobic polymer is different from that of the matrix polymer.

The present invention also provides a process for the preparation of said microparticles. It additionally provides a method of use that comprises application of a solid or liquid personal care formulation having an effective brightening amount of said shatter-resistant microparticles containing at least one encapsulated fluorescent whitening agent to at least a part of the body, for example to the surface of the skin.

The encapsulated particles according to the first aspect of the invention and the products resulting from the process according to the second aspect of the invention provide enhanced visual performance in cosmetic formulations. The shatter-resistant microparticles are able to both scatter and reemit white light in a diffuse manner in order to reduce the visual appearance and perception of skin imperfections, such as shadows, skin discolorations, wrinkles and cellulite when applied to at least a part of the body, for example to the surface of the skin. Furthermore the polymer matrix does not allow the encapsulated fluorescent whitening agent to be released even under prolonged use.

### DETAILED DESCRIPTION OF THE INVENTION

Microparticulate particles comprising at least one fluorescent whitening agent entrapped within a transparent or translucent matrix polymer and having transparent or translucent secondary particles distributed throughout the matrix, wherein the matrix polymer has been formed from a blend of monomers comprising a first monomer which is an ethylenically unsaturated ionic monomer and a second monomer which is an ethylenically unsaturated hydrophobic monomer which is capable of forming a homopolymer of glass transition temperature in excess of 50° C., in which the secondary particles comprise a hydrophobic polymer that has been formed from an ethylenically unsaturated hydrophobic monomer which is capable of forming a homopolymer of glass transition temperature in excess of 50° C. and optionally other monomers, which hydrophobic polymer is different from the matrix polymer, may be obtained by a process which comprises the steps,
A) providing an aqueous phase of a polymeric salt formed from a monomer blend which comprises the first and second monomers (the matrix polymer) plus the FWA,
B) forming the secondary particles in the aqueous phase or combining the secondary particles with the aqueous phase,
C) forming a dispersion comprising the aqueous phase in a water immiscible liquid phase, which preferably comprises an amphipathic polymeric stabilizer to form an emulsion, and
D) subjecting the dispersion to dehydration wherein water is evaporated from the aqueous particles thereby forming solid particles comprising the secondary particles and the FWA distributed throughout the matrix polymer.

Preferably the first monomer used to form the matrix polymer is a salt of a volatile counterion component. During the dehydration step (D) the volatile counterion component of the salt is desirably evaporated. Consequently, during the distillation stage the matrix polymer would be converted to its free acid or free base form.

The microparticles useful in the present invention comprise at least one fluorescent whitening agent. In the process of preparing the microparticles it is particularly desirable for the fluorescent whitening agent to be dissolved or finely dispersed in the aqueous phase of step A) above so that it can become substantially uniformly distributed throughout the transparent or translucent matrix polymer.

The polymeric products can be further enhanced if the matrix polymer is cross-linked. This cross-linking can be as a result of including a cross-linking step in the process. This can be achieved by including self cross-linking groups in the polymer, for instance monomer repeating units carrying a methylol functionality.

Preferably though the cross-linking is achieved by including a cross-linking agent with the aqueous phase polymer. The cross-linking agents are generally compounds which react with functional groups on the polymer chain. For instance when the polymer chain contains anionic groups, suitable cross-linking agents may be aziridines, diepoxides, carbodiamides, silanes or a multivalent metal, for instance aluminum or zirconium. One preferred cross-linking agent is ammonium zirconium carbonate. Another particularly preferred class of cross-linking agents includes compounds which form covalent bonds between polymer chains, for instance silanes or diepoxides.

The cross-linking process desirably occurs during the dehydration step. Thus where a cross-linking agent is included, it will generally remain dormant until the dehydration is started.

It has been found that polymers formed from the special combination of hydrophobic monomer that are capable of forming a homopolymer of glass transition temperature in excess of 50° C., preferably greater than 60° C., more preferably greater than 80° C. exhibit considerably improved performance in regard to the impermeability to the FWA. By hydrophobic monomer is meant that the monomer has a solubility in water of less than 5 g per 100 ml water.

The glass transition temperature (T_{g}) for a polymer is defined in the Encyclopedia of Chemical Technology, Volume 19, fourth edition, page 891, as the temperature below which (1) the transitional motion of entire molecules and (2) the coiling and uncoiling of 40 to 50 carbon atom segments of chains are both frozen. Thus below its T_{g} a polymer would not exhibit flow or rubber elasticity.

The T_{g} of a polymer may be determined using Differential Scanning Calorimetry (DSC) by methods well known in the art.

Generally the average particle size diameter of the particles is less than about 200 microns. Usually the average particle size diameter tends to be smaller, for instance less than 150 or 100 microns, and typically the average particle diameter will be between 750 nanometers and 200 microns.

Preferably the average particle size diameter is in the range 10 to 150 microns and especially between 20 and 100 microns. Average particle size is determined by a Coulter particle size analyzer according to standard procedures well documented in the literature.

Without being limited to theory it is believed that the particular combination of an ionic monomer and a hydrophobic monomer provides polymers with the right degree of hydrophilicity and hardness that seems to be responsible for the improvements in impermeability to the FWA.

Specific examples of said hydrophobic monomers include styrene, methyl methacrylate, tertiary butyl methacrylate, phenyl methacrylate, cyclohexyl methacrylate and isobornyl methacrylate.

It has been found that it is not possible to replace the hydrophobic monomers with ethylenically unsaturated carboxylic acid esters that are not capable of forming a hompolymer that has a glass transition temperature (T_{g}) of at least 50° C. without adversely increasing the permeability of the polymer. Preferably still the T_{g} should be at least 60° C. or even at least 80° C. For instance substituting the hydrophobic monomer of the present invention by other (meth)acrylic esters, for instance 2-ethylhexyl acrylate would be unsuitable. Best results are generally obtained by use of monomers which are capable of forming polymers of very high T_{g}.

Therefore less preferred products would be produced using ethyl acrylate or propyl acrylate as the hydrophobic monomer.

The ionic monomer may contain either anionic or cationic groups or alternatively may be potentially ionic, for instance in the form of an acid anhydride. Preferably the ionic monomer is an ethylenically unsaturated anionic or potentially anionic monomer. Suitable anionic monomers include (meth)acrylic acid, maleic acid, maleic anhydride, itaconic acid anhydride, crotonic acid, (meth)allyl sulfonic acid, vinyl sulfonic acid and 2-acrylamido-2-methyl propane sulfonic acid. Preferred anionic monomers are carboxylic acids or acid anhydrides.

When the ionic monomer is anionic, for instance a carboxylic acid or anhydride, the volatile counterion may be ammonia or a volatile amine component. Thus the polymer may be produced in free acid form and then neutralized with an aqueous solution of ammonium hydroxide or a volatile amine, for instance ethanolamine.

Alternatively the polymer may be prepared by copolymerizing the ammonium or volatile amine salt of an anionic monomer with the hydrophobic monomer.

Generally the matrix polymer may be prepared by any suitable polymerization process. For instance the polymer can be conveniently prepared by aqueous emulsion polymerization as described for instance in EP-A-697423 or U.S. Pat. No. 5,070,136. The polymer can then be neutralized by the addition of an aqueous solution of ammonium hydroxide or a volatile amine.

In a typical polymerization process a blend of hydrophobic monomer and anionic monomer is emulsified into an aqueous phase which contains a suitable amount of emulsifying agent. Typically the emulsifying agent may be any commercially available emulsifying agent suitable for forming an aqueous emulsion.

Desirably these emulsifying agents will tend to be more soluble in the aqueous phase than in the water-immiscible monomer phase and thus will tend to exhibit a high hydrophilic lipophilic balance (HLB). Emulsification of the monomer may be effected by known emulsification techniques, including subjecting the monomer/aqueous phase to vigorous stirring or shearing or alternatively passing the monomer/aqueous phase through a screen or mesh. Polymerization may then be effected by use of suitable initiator systems, for instance a UV initiator or thermal initiator. A suitable technique of initiating the polymerization would be to elevate the temperature of the aqueous emulsion of monomer to above 70 or 80° C. and then add between 50 and 1000 ppm of ammonium persulfate by weight of monomer.

Generally the matrix polymer has a molecular weight of up to 200,000 (Determined by GPC using standard industrial parameters). Preferably the polymer has a molecular weight of below 50,000, for instance 2,000 to 20,000. It is important that the matrix polymer is transparent or translucent.

Usually the optimum molecular weight for the matrix polymer is around 8,000 to 12,000. Typically the monomer blend may contain at least 50% by weight hydrophobic monomer, the remainder being made up of anionic monomer. Generally though the hydrophobic monomer will be present in amounts of at least 60% by weight.

Preferred compositions contain between 65 and 90% by weight of hydrophobic repeating units, for instance around 70 or 75%.

A particularly preferred matrix polymer is a copolymer of styrene with ammonium acrylate. More preferably this polymer is used when the process employs a cross-linking agent, which is especially ammonium zirconium carbonate.

In an alternative version of the process of the present invention the ionic monomer may be cationic or potentially cationic, for instance an ethylenically unsaturated amine. In this form of the invention the volatile counterionic component is a volatile acid component. Thus in this form of the invention the matrix polymer can be formed in an analogous way to the aforementioned anionic matrix polymer, except that the anionic monomer is replaced by a cationic or potentially cationic monomer. Generally where the polymer is prepared in the form of a copolymer of a free amine and hydrophobic monomer, it is neutralized by including a suitable volatile acid, for instance acetic acid, formic acid or even carbonic acid. Preferably the polymer is neutralized by a volatile carboxylic acid. The amount of cationic or potentially cationic monomer to hydrophobic monomer to employ is generally the same as for the aforementioned anionic monomer.

Step C) of the process of the present invention involves dispersing an aqueous solution of matrix polymer containing a fluorescent whitening agent into a water immiscible liquid. Typically the water immiscible liquid is an organic liquid or blend of organic liquids. A preferred organic liquid is a paraffin oil. In one embodiment the organic liquid is a mixture of a non-volatile paraffin oil and a volatile paraffin oil. The two oils may be used in equal proportions by weight, but generally it is preferred to use the non-volatile oil in excess, for instance greater than 50 to 75 parts by weight of the non-volatile oil to 25 to less than 50 parts by weight of the volatile oil.

In the process according to the second aspect of the invention it is desirable to include a polymeric amphipathic stabilizer in the water immiscible liquid. The amphipathic stabilizer may be any suitable commercially available amphipathic stabilizer, for instance HYPERMER® (available from ICI). Suitable stabilizers also include the stabilizers described in WO-A-97/24179.

Although it is possible to include other stabilizing materials in addition to the amphipathic stabilizer, such as surfactants, it is generally preferred that the sole stabilizing material be the amphipathic stabilizer.

In the process of the present invention the dehydration step can be achieved by any convenient means. Desirably dehydration can be effected by subjecting the dispersion in oil to vacuum distillation. Generally this will require elevated temperatures, for instance temperatures of 30° C. or higher. Although it may be possible to use much higher temperatures, e.g. 80 to 90° C., it is generally preferred to use temperatures of below 60 or 70° C.

Instead of vacuum distillation it may be desirable to effect dehydration by spray drying. Suitably this can be achieved by the spray drying process described in WO-A-97/34945.

The dehydration step removes water from the aqueous solution of the matrix polymer and also the volatile counterion component, resulting in a dry polymer matrix which is insoluble and non-swellable in water, containing therein the FWA which is distributed throughout the polymeric matrix.

Fluorescent whitening agents are substances that absorb light in the invisible ultraviolet region of the spectrum and reemit it in the visible portion of the spectrum, particularly in the blue to blue-violet wavelengths. This provides added brightness and can offset the darkening in areas of a substrate such as skin due to crevices or wrinkles.

The microparticles of the invention have one or more fluorescent whitening agents entrapped within.

The choice of the fluorescent whitening agent used in the present invention is not critical. It can be oil or water soluble and may be selected from a wide range of chemical classes such as 4,4′-bis-(triazinylamino)-stilbene-2,2′-disulfonic acids, 4,4′-bis-(triazol-2-yl)stilbene-2,2′-disulfonic acids, 4,4′-dibenzofuranyl-biphenyls, 4,4′-(diphenyl)-stilbenes, 4,4′-distyryl-biphenyls, 4-phenyl-4′-benzoxazolyl-stilbenes, stilbenyl-naphthotriazoles, 4-styryl-stilbenes, bis-(benzoxazol-2-yl) derivatives, bis-(benzimidazol-2-yl) derivatives, coumarins, pyrazolines, naphthalimides, triazinyl-pyrenes, 2-styryl-benzoxazole or -naphthoxazoles, benzimidazole-benzofurans and oxanilides. Mixtures thereof of fluorescent whitening agents of the same or different chemical classes may be employed.

Preferred 4,4′-bis-(triazinylamino)-stilbene-2,2′-disulfonic acids include those having the formula: 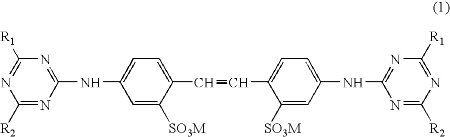
in which R₁ and R₂, independently, are phenyl, mono- or disulfonated phenyl, phenylamino, mono- or disulfonated phenylamino, morpholino, —N(CH₂CH₂OH)₂, —N(CH₃)(CH₂CH₂OH), —NH₂, —N(C₁-C₄alkyl)₂, —OCH₃, —Cl, —NH—CH₂CH₂SO₃H, CH₂CH₂OH or ethanolaminopropionic acid amide; and M is H, Na, Li, K, Ca, Mg, ammonium, or ammonium that is mono-, di-, tri- or tetra-substituted by C₁-C₄alkyl, C₁-C₄hydroxyalkyl or a mixture thereof. Preferably M is Na, Li or K.

Especially preferred compounds of formula (1) include those in which each R₁ is 2,5-disulfo-phenyl and each R₂ is morpholino, —N(C₂H₅)₂, —N(CH₂CH₂OH)₂ or ethanolaminopropionic acid amide; or each R₁ is 3-sulfophenyl and each R₂ is NH(CH₂CH₂OH) or N(CH₂CH₂OH)₂; or each R₁ is 4-sulfophenyl and each R₂ is N(CH₂CH₂OH)₂, N(CH₂CHOHCH₃)₂, morpholino, or ethanolaminopropionic acid amide; or each R₁ is phenylamino and each R₂ is morpholino, NH(CH₂CH₂OH), N(CH₂CH₂OH)CH₃, N(CH₂CH₂OH)₂ or ethanolaminopropionic acid amide, and, in each case, the sulfo group is SO₃M in which M is sodium.

The compounds of the formulae 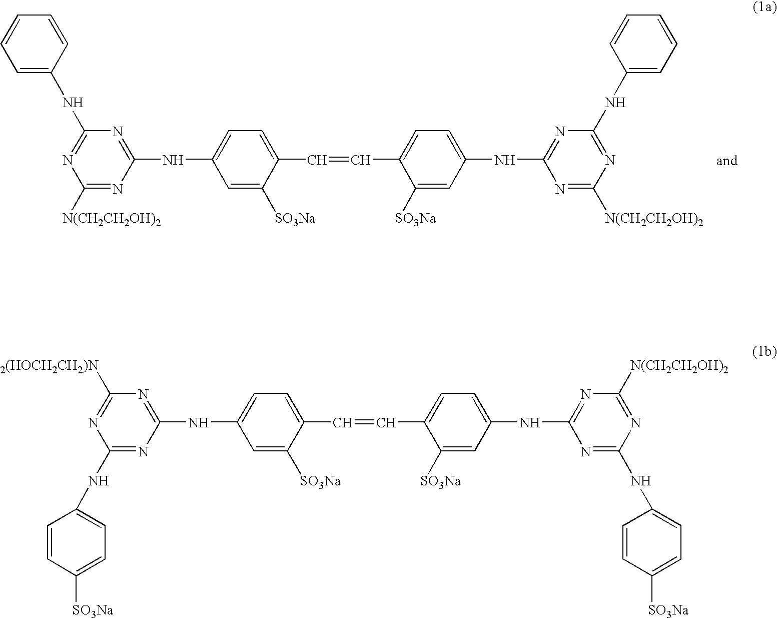
are particularly especially preferred.

Preferred 4,4′-bis-(triazol-2-yl)stilbene-2,2′-disulfonic acids are those having the formula: 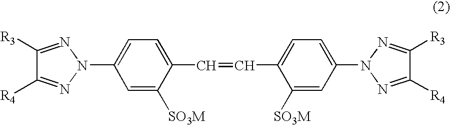
in which R₃ and R₄, independently, are H, C₁-C₄alkyl, phenyl or monosulfonated phenyl; and M is H, Na, Li, K, Ca, Mg, ammonium, or ammonium that is mono-, di-, tri- or tetra-substituted by C₁-C₄alkyl, C₁-C₄-hydroxyalkyl or a mixture thereof. Preferably M is Na, Li or K.

Especially preferred compounds of formula (2) are those in which R₃ is phenyl, R₄ is H and M is sodium.

Preferred 4,4′-dibenzofuranyl-biphenyls include those of the formula: 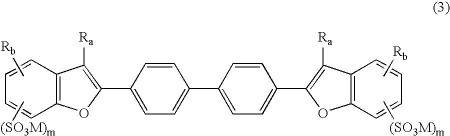
in which Rₐ and R_{b}, independently, are H or C₁-C₄alkyl, and M is H, Na, Li, K, Ca, Mg, ammonium, or ammonium that is mono-, di-, tri- or tetra-substituted by C₁-C₄alkyl, C₁-C₄-hydroxyalkyl or a mixture thereof. Preferably M is Na, Li or K.

Especially preferred is the compound of the formula:

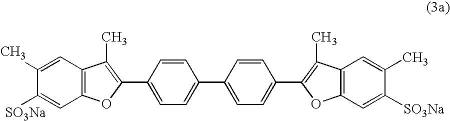

Preferably, the 4,4′-distyryl-biphenyls used are those of the formula: 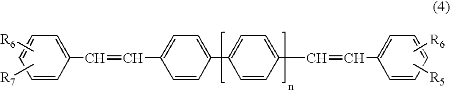
in which R₅ and R₆, independently, are H, SO₃M, SO₂N(C₁-C₄alkyl)₂, O—(C₁-C₄alkyl), CN, Cl, COO(C₁-C₄alkyl), CON(C₁-C₄alkyl)₂ or O(CH₂)₃N^{⊕}(CH₃)₂An⁻, in which M is H, Na, Li, K, Ca, Mg, ammonium, or ammonium that is mono-, di-, tri- or tetra-substituted by C₁-C₄alkyl, C₁-C₄-hydroxyalkyl or a mixture thereof, An⁻ is an anion of an organic or inorganic acid or a mixture thereof, and n is 1. Preferably M is Na, Li or K and An⁻ is a formate, acetate, propionate, gicolate, lactate, acrylate, methanephosphonate, phosphite, dimethyl or diethyl phosphite anion, or a mixture thereof.

Especially preferred compounds of formula (4) include those in which each R₆ is H, each R₅ is a 2-SO₃M group in which M is sodium or each R₅ is O(CH₂)₃N^{⊕}(CH₃)₂An⁻, in which An⁻ is acetate. Especially particularly preferred is the compound of the formula (4a)

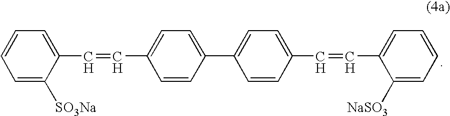

Preferred 4-phenyl-4′-benzoxazolyl-stilbenes have the formula: 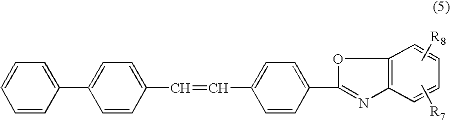
in which R₇ and R₈, independently, are H, Cl, C₁-C₄alkyl or SO₂—C₁-C₄alkyl.

Preferably, the stilbenyl-naphthotriazoles used are those of the formula: 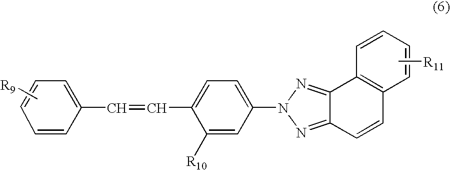
in which R₉ is H or Cl; R₁₀ is SO₃M, SO₂N(C₁-C₄alkyl)₂, SO₂O-phenyl or CN; R₁₁ is H or SO₃M; and M is H, Na, Li, K, Ca, Mg, ammonium, or ammonium that is mono-, di-, tri- or tetra-substituted by C₁-C₄alkyl, C₁-C₄hydroxyalkyl or a mixture thereof. Preferably M is Na, Li or K.

Especially preferred compounds of formula (6) are those in which R₉ and R₁₁ are H and R₁₀ is 2-SO₃M in which M is Na.

Preferably, the 4-styryl-stilbenes used include those of the formula: 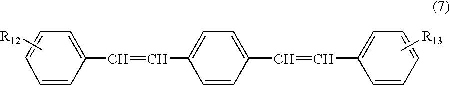
in which R₁₂ and R₁₃, independently, are H, SO₃M, SO₂N(C₁-C₄alkyl)₂, O—(C₁-C₄alkyl), CN, Cl, COO(C₁-C₄alkyl), CON(C₁-C₄alkyl)₂ or O(CH₂)₃N^{⊕}(CH₃)₂An⁻ in which An⁻ is an anion of an organic or inorganic acid, in particular a formate, acetate, propionate, gicolate, lactate, acrylate, methanephosphonate, phosphite, dimethyl or diethyl phosphite anion, or a mixture thereof and M is H, Na, Li, K, Ca, Mg, ammonium, or ammonium that is mono-, di-, tri- or tetra-substituted by C₁-C₄alkyl, C₁-C₄hydroxyalkyl or a mixture thereof. Preferably M is Na, Li or K.

Especially preferred compounds of formula (7) are those in which each of R₁₂ and R₁₃ is 2-cyano or 2-SO₃M in which M is sodium or O(CH₂)₃N^{⊕}(CH₃)₂An⁻ in which An⁻ is acetate.

Preferred bis-(benzoxazol-2-yl) derivatives include those of the formula: 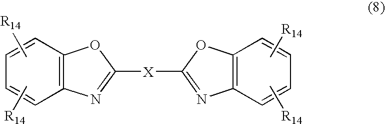
in which each R₁₄, independently, is H, C(CH₃)₃, C(CH₃)₂-phenyl, C₁-C₄alkyl or COO—C₁-C₄alkyl, and X is —CH═CH— or a group of the formula:

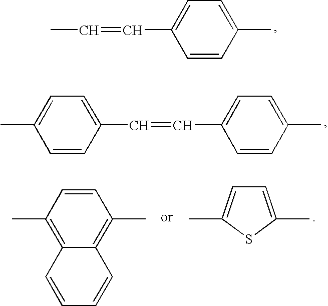

Especially preferred compounds of formula (8) are those in which each R₁₄ is H and X is 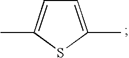
or one group R₁₄ in each ring is 2-methyl and the other R₁₄ is H and X is —CH═CH—; or one group R₁₄ in each ring is 2-C(CH₃)₃ and the other R₁₄ is H and X is

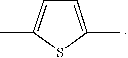

Preferred bis-(benzimidazol-2-yl) derivatives include those of the formula: 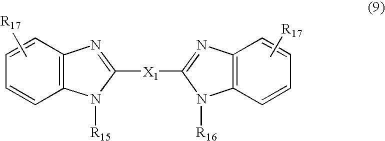
in which R₁₅ and R₁₆, independently, are H, C₁-C₄alkyl or CH₂CH₂OH; R₁₇ is H or SO₃M; X₁ is —CH═CH— or a group of the formula: 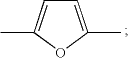
and M is H, Na, Li, K, Ca, Mg, ammonium, or ammonium that is mono-, di-, tri- or tetra-substituted by C₁-C₄alkyl, C₁-C₄ hydroxyalkyl or a mixture thereof. Preferably M is Na, Li or K.

Especially preferred compounds of formula (9) are those in which R₁₅ and R₁₆ are each H, R₁₇ is SO₃M in which M is sodium and X₁ is —CH═CH—.

Preferred coumarins include those of the formula: 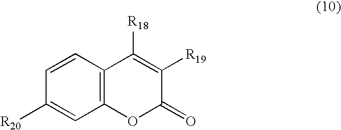
in which R₁₈ is H, C₁ or CH₂COOH, R₁₉ is H, phenyl, COO—C₁-C₄alkyl or a group of the formula: 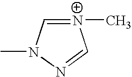
R₂₀ is O—C₁-C₄alkyl, N(C₁-C₄alkyl)₂, NH—CO—C₁-C₄alkyl or a group of the formula: 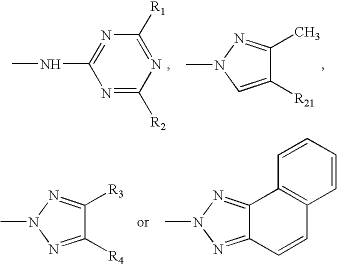
in which R₁ and R₂, independently, are phenyl, mono- or disulfonated phenyl, phenylamino, mono- or disulfonated phenylamino, morpholino, —N(CH₂CH₂OH)₂, —N(CH₃)(CH₂CH₂OH), —NH₂, —N(C₁-C₄alkyl)₂, —OCH₃, —Cl, —NH—CH₂CH₂SO₃H or —NH—CH₂CH₂OH, R₃ and R₄, independently, are H, C₁-C₄alkyl, phenyl or monosulfonated phenyl and R₂₁ is H, C₁-C₄alkyl or phenyl.

Especially preferred compounds of formula (10) are those having the formula:

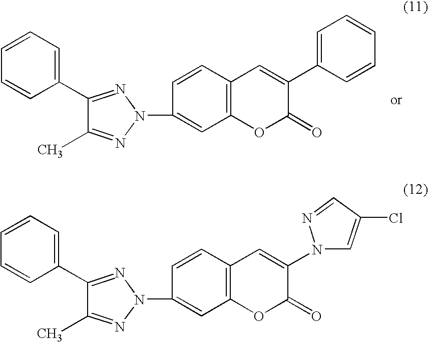

Preferably, the pyrazolines used are those having the formula: 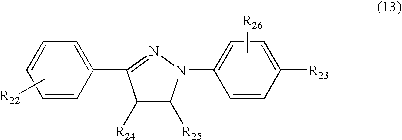
in which R₂₂ is H, Cl or N(C₁-C₄alkyl)₂, R₂₃ is H, C₁, SO₃M, SO₂NH₂, SO₂NH—(C₁-C₄alkyl), COO—C₁-C₄alkyl, SO₂—C₁-C₄alkyl, SO₂NHCH₂CH₂CH₂N^{⊕}(CH₃)₃ or SO₂CH₂CH₂N^{⊕}H(C₁-C₄alkyl)₂ An⁻, R₂₄ and R₂₅ are the same or different and each is H, C₁-C₄alkyl or phenyl, R₂₆ is H or Cl, An⁻ is an anion of an organic or inorganic acid, and M is H, Na, Li, K, Ca, Mg, ammonium, or ammonium that is mono-, di-, tri- or tetra-substituted by C₁-C₄alkyl, C₁-C₄hydroxyalkyl or a mixture thereof. Preferably M is Na, Li or K.

Especially preferred compounds of formula (13) are those in which R₂₂ is Cl, R₂₃ is SO₂CH₂CH₂N^{⊕}(C₁-C₄alkyl)₂ An⁻ in which An⁻ is phosphite and R₂₄, R₂₅ and R₂₆ are each H; or those having the formula:

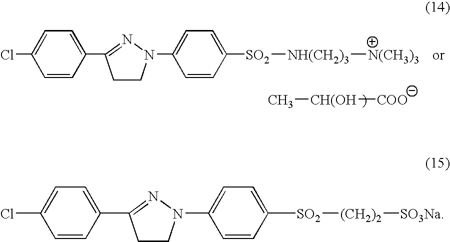

Preferred naphthalimides are those of the formula: 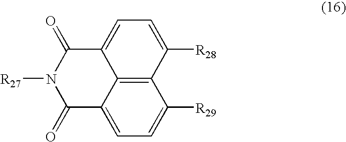
in which R₂₇ is C₁-C₄alkyl or CH₂CH₂CH₂N^{⊕}(CH₃)₃ An⁻ in which An⁻ is an anion of an organic or inorganic acid, R₂₈ and R₂₉, independently, are O—C₁-C₄-alkyl, SO₃M or NH—CO—C₁-C₄alkyl; and M is H, Na, Li, K, Ca, Mg, ammonium, or ammonium that is mono-, di-, tri- or tetra-substituted by C₁-C₄alkyl, C₁-C₄hydroxyalkyl or a mixture thereof. Preferably M is Na, Li or K.

Especially preferred compounds of formula (16) are those having the formula:

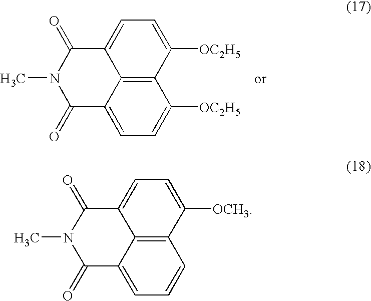

Preferred 2-styryl-benzoxazole- or -naphthoxazole derivatives include those having the formula: 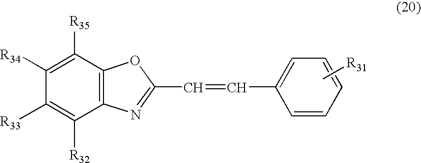
in which R₃₁ is CN, Cl, COO—C₁-C₄alkyl or phenyl; R₃₂ and R₃₃ are the atoms required to form a fused benzene ring or R₃₃ and R₃₅, independently, are H or C₁-C₄alkyl; and R₃₄ is H, C₁-C₄alkyl or phenyl.

Preferred benzimidazole-benzofuran derivatives include those having the formula: 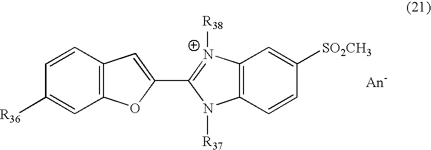
in which R₃₆ is C₁-C₄alkoxy; R₃₇ and R₃₈, independently, are C₁-C₄alkyl; and An⁻ is an anion of an organic or inorganic acid.

A particularly preferred compound of formula (21) is that in which R₃₆ is methoxy, R₃₇ and R₃₈ are each methyl and An⁻ is methane sulfonate.

Preferred oxanilide derivatives include those having the formula: 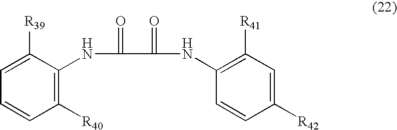
in which R₃₉ is C₁-C₄alkoxy, R₄₁ is C₁-C₄alkyl, C₁-C₄alkyl-SO₃M or C₁-C₄alkoxy-SO₃M in which M is H, Na, Li, K, Ca, Mg, ammonium, or ammonium that is mono-, di-, tri- or tetra-substituted by C₁-C₄alkyl, C₁-C₄hydroxyalkyl or a mixture thereof, preferably Na, Li or K, and R₄₀ and R₄₂ are the same and each is hydrogen, tert. butyl or SO₃M, in which M is as defined for R₄₁.

Compounds of the above formulae are known per se and can be prepared by known methods.

It is noted that fluorescent whitening agents may exhibit a green or bluish cast in concentrated form. This can be counteracted by the use of appropriate levels of mixtures of fluorescent whitening agents, particularly mixtures which contain fluorescent whitening agents having a more reddish cast. Therefore mixtures of fluorescent whitening agents of the same or different chemical classes may be advantageously employed.

It has been found that the polymeric microparticles described above exhibit improved shatter resistance in combination with improved visual performance. Furthermore the polymer matrix does not allow the entrapped fluorescent whitening agent to be released even under prolonged use. It is particularly desirable to provide particles in which the fluorescent whitening agent is distributed throughout the matrix polymer and wherein the matrix polymer is impermeable to the fluorescent whitening agent.

The personal care or cosmetic composition according to the invention comprises from 0.0001 to 10% by weight, for example from 0.001 to 8% by weight, and especially from 0.005 to 5% by weight based on the total weight of the composition, of the fluorescent whitening agent-containing polymeric microparticles described above as well as a cosmetically tolerable carrier or adjuvant which is other than, or in addition to water. While water is cosmetically tolerable, and in most instances will also be present, the phrase “a cosmetically tolerable carrier or adjuvant” is intended to refer to at least one substance other than water that is customarily employed in personal care or cosmetic compositions.

Polymeric microparticles described above having average diameters of 0.1 to 60 microns are preferred, for example 5 to 40 and especially 10 to 30 microns.

Depending on the intended use, the preferred average diameters will vary. For example one embodiment of this invention may be a liquid facial cosmetic formulation comprising the fluorescent whitening agent-containing polymeric microparticles described above and having a preferred particle size range of between 10 and 30 microns. Another embodiment may be a lipstick formulation comprising the fluorescent whitening agent-containing polymeric microparticles described above having preferred particle sizes of between 1 and 10 microns.

The personal care or cosmetic preparation according to the invention may be formulated as a water-in-oil or oil-in-water emulsion, as an alcoholic or alcohol-containing formulation, as a vesicular dispersion of an ionic or non-ionic amphiphilic lipid, as a gel, or a solid stick. Preferably the cosmetic preparation is in the form of a liquid.

As a water-in-oil or oil-in-water emulsion, the cosmetically tolerable adjuvant contains preferably from 5 to 50% of an oily phase, from 5 to 20% of an emulsifier and from 30 to 90% water. The oily phase may contain any oil suitable for cosmetic formulations, e.g. one or more hydrocarbon oils, a wax, a natural oil, a silicone oil, a fatty acid ester or a fatty alcohol. Examples are mineral oil, castor oil, cyclomethicone, dimethicone, dimethicone copolyol, phenyl trimethicone, trimethyl pentaphenyl trisiloxane, caprylic/capric triglyceride, isostearyl stearoyl stearate, octyidodecyl erucate, triisostearyl citrate, triisostearyl trilinoleate, pentaerythrityl tetraisononanoate, isopropyl myristate, isopropyl palmitate, octyl palmitate, diisostearyl malate, diethyl sebacate and diisopropyl adipate.

Cosmetic liquids may contain mono- or polyols such as ethanol, isopropanol, propylene glycol, hexylene glycol, glycerol or sorbitol.

Cosmetic formulations according to the invention may be contained in a wide variety of cosmetic preparations. Especially the following preparations, for example, come into consideration:
shaving preparations, e.g. aftershave lotions or aftershave creams;
skin-care preparations, e.g. skin emulsions, multi-emulsions or skin oils and body powders;
cosmetic personal care preparations, e.g. facial make-up in the form of lipsticks, day creams, facial lotions, and creams; and
light-protective preparations, such as sun tan lotions, creams and oils, sun blocks and pretanning preparations.

Depending upon the form of the personal care or cosmetic preparation, it will comprise, in addition to the polymeric microparticles described above, further constituents, for example sequestering agents, non-encapsulated or encapsulated colorants, perfumes, thickening or solidifying (consistency regulator) agents, emollients, non-encapsulated or encapsulated UV absorbers, skin-protective agents, antioxidants and preservatives.

The term “perfume” or “fragrance” as used herein refers to odoriferous materials which are able to provide a pleasing fragrance to fabrics, and encompasses conventional materials commonly used in cosmetic compositions to counteract a malodor in such compositions and/or provide a pleasing fragrance thereto. The perfumes are preferably in the liquid state at ambient temperature, although solid perfumes are also useful, particularly cyclodextrin/perfume inclusion complexes for controlled release. Included among the perfumes contemplated for use herein are materials such as aldehydes, ketones, esters and the like which are conventionally employed to impart a pleasing fragrance to liquid and solid personal care or cosmetic compositions. Naturally occurring plant and animal oils are also commonly used as components of perfumes. Accordingly, the perfumes useful for the present invention may have relatively simple compositions or may comprise complex mixtures of natural and synthetic chemical components, all of which are intended to provide a pleasant odor or fragrance when applied to fabrics. The perfumes used in personal care or cosmetic compositions are generally selected to meet the normal requirements of odor, stability, price and commercial availability. The term “fragrance” is often used herein to signify a perfume itself, rather than the aroma imparted by such perfume.

As a further customary additive, the personal care or cosmetic compositions may also comprise at least one component capable of sequestering properties, that is a component which Sequestering agents act to sequester (chelate) metal ions. Said sequestering agents may be present at a level of up to 0.5%, more preferably from 0.005% to 0.25%, most preferably from 0.01% to 0.1 wt-%, based on the total weight of the personal care or cosmetic composition.

Compositions according to the invention may be prepared by physically blending polymeric microparticles as described above containing one or more fluorescent whitening agents into personal care formulations by methods which are well known in the art. The examples illustrate several such methods.

The shatter-resistant microparticles according to the invention are able to both scatter and reemit white light in a diffuse manner in order to reduce the visual appearance and perception of skin imperfections, such as shadows, skin discolorations, wrinkles and cellulite when applied to at least a part of the body, for example to the surface of the skin. Hence the present invention additionally relates to a method of masking or reducing the appearance of skin imperfections, which comprises applying a solid or liquid personal care or cosmetic formulation having an effective amount of shatter-resistant polymeric microparticles according to the invention to the surface of the skin.

In one embodiment of the method, the personal care or cosmetic formulation comprises from 0.0001 to 10% by weight, for example from 0.001 to 8% by weight, and especially from 0.005 to 5% by weight based on the total weight of the formulation, of the polymeric microparticles described above.

In one embodiment of the method, the personal care or cosmetic composition comprises a blend of polymeric microparticles as described above containing different microencapsulated fluorescent whitening agents that are individually provided in separate polymeric matrix materials. In another, the personal care or cosmetic composition comprises polymeric microparticles as described above containing a blend of at least two different fluorescent whitening agents that are embedded in a single polymeric matrix material.

In one embodiment of the method, the personal care or cosmetic composition is formulated as a water-in-oil or oil-in-water emulsion, as an alcoholic or alcohol-containing formulation, as a vesicular dispersion of an ionic or non-ionic amphiphilic lipid, as a gel, or a solid stick.

In various embodiments of the method, the personal care or cosmetic composition is in the form of a shaving preparation, a skin-care preparation, a cosmetic personal care preparation or a light-protective preparation.

The following examples describe certain embodiments of this invention, but the invention is not limited thereto. It should be understood that numerous changes to the disclosed embodiments could be made in accordance with the disclosure herein without departing from the spirit or scope of the invention. These examples are therefore not meant to limit the scope of the invention. Rather, the scope of the invention is to be determined only by the appended claims and their equivalents. In these examples all parts given are by weight unless otherwise indicated.

### EXAMPLE 1

Shatter-Resistant FWA Micro-beads Comprising 1% FWA by Weight are Prepared as Follows:—

An aqueous phase is prepared by diluting 300 g of 46% polymer microemulsion containing 14 weight % of styrene-acrylic acid copolymer (65/35 weight % monomers ratio, molecular weight 6,000) and 32 weight % styrene-methyl methacrylate copolymer (70/30 weight % monomers ratio, molecular weight 200,000) with 150 g of water and then milling 1.5 g of disodium distyrylbiphenyl disulfonate powder of the formula (4a) (Tinopal CBS-X from Ciba Specialty Chemicals) with a high speed mixer. To the resulting aqueous FWA dispersion is added 20 g of 50% aqueous solution of ammonium zirconium carbonate.

Separately, an oil phase is prepared by diluting 80 g of 20% an amphipathic stabilizer (90 weight % stearyl methacrylate, 10 weight % methacrylic acid copolymer, molecular weight 40,000) with 1200 g of Isopar G solvent (available from Exxon Mobil). The above aqueous phase is added to this oil phase under a high shear homogenizer to form a water-in-oil emulsion having a mean aqueous droplet particle diameter of 10 microns. The formed emulsion is transferred to a 4-litre reactor set up for vacuum distillation. The emulsion is warmed to 60° C. and then subjected to vacuum distillation to remove a water/Isopar G solvent mixture. Vacuum distillation is continued to 100° C. until no further water is collected in the distillate. Next, the reactor contents are held for further 1 hour to complete the crosslinking reaction between the zirconium crosslinker and the carboxylated supported resin.

After this heat treatment step, the reactor contents are cooled to 25° C. and the fluorescent micro-beads formed are isolated by filtration and oven drying at 80° C. The product is washed with deionized water and dried.

The final product is free flowing fluorescent micro-beads having a mean particle size diameter of 10 microns.

### FORMULATION EXAMPLE 1

### Oil-in-Water Cream Containing 0.04% FWA

| | | Control | FWA Test |
|---|---|---|---|
| | | Cream | Cream |
| | | Amount | Amount |
| Phase | Ingredient | [wt-%] | [wt-%] |
| A | Deionized Water | 82.95 | 80.95 |
| | Hydroxyethylcellulose | 0.70 | 0.70 |
| | Methylparaben, USP | 0.30 | 0.30 |
| B | C₁₂-C₁₅ Alkyl Octanoate | 3.00 | 3.00 |
| | Trioctyldodecyl Citrate | 2.00 | 2.00 |
| | Ethylhexyl Palmitate | 3.00 | 3.00 |
| | Glyceryl Stearate | 1.00 | 1.00 |
| | Stearic Acid | 2.00 | 2.00 |
| | Sorbitan Oleate | 0.80 | 0.80 |
| | Polysorbate 80 | 0.15 | 0.15 |
| | Propylparaben, USP | 0.10 | 0.10 |
| C | Deionized Water | 2.00 | 2.00 |
| | Triethanolamine | 0.70 | 0.70 |
| D | Encapsulated FWA of Example 1 | 0.00 | 2.00 |
| E | Deionized Water | 1.00 | 1.00 |
| | Diazolidinyl Urea | 0.30 | 0.30 |

In a suitable vessel the water and the hydroxyethylcellulose of phase A are mixed using a homogenizer for 60 minutes and heated to 75-80° C. Then the methyl paraben of phase A is added and mixed for about 5 minutes, maintaining the same temperature as above. The ingredients of phase B are premelted at 75-80° C. and mixed in a separate vessel. When a uniform liquid solution is obtained phase B is added to phase A using high-speed homogenizer. The emulsion is homogenized for 15-20 minutes at 75-80° C. Phase C is added to the vessel while using the homogenizer. The heating process is stopped and the mixture is allowed to gradually cool down. At 60-65° C. phase D is added using a lightning mixer. At 45° C. phase E is added to the vessel. The mixing is stopped when room temperature is achieved.

This results an O/W cream with good overall properties.

### FORMULATION EXAMPLE 2

### Liquid Makeup—Oil-in-Water Foundation

| Phase | Ingredient | Amount [wt-%] |
|---|---|---|
| A | Deionized Water | 53.88 |
| | Sodium Hydroxide 10% solution | 1.30 |
| | Dimethicone Copolyol | 0.10 |
| B | 80% Titanium Dioxide/Talc | 7.50 |
| | 80% Yellow Iron Oxide/Talc | 2.25 |
| | 80% Red Iron Oxide/Talc | 1.38 |
| | 80% Black Iron Oxide/Talc | 0.25 |
| | Talc | 0.72 |
| C | Butylene Glycol | 4.00 |
| | Magnesium Aluminum Silicate | 1.00 |
| D | Butylene Glycol | 2.00 |
| | Cellulose Gum | 0.10 |
| E | Methylparaben | 0.10 |
| F | Di-PPG-3 Myristyl Ether Adipate | 14.00 |
| | Dioctyl Maleate | 4.00 |
| | Steareth-10 | 2.00 |
| | Steareth-2 | 0.50 |
| | Cetyl Alcohol | 0.62 |
| | Dicetyl Phosphate, Ceteth-20 Phosphate, | 4.00 |
| | Cetearyl Alcohol | |
| | Propylparaben | 0.10 |
| G | Encapsulated FWA of Example 1 | 0.02 |
| H | DMDM Hydantoin | 0.18 |
| | Total | 100.00 |

In a suitable vessel phase A is heated to 75-80° C. and mixed well using a homogenizer. Pre-ground phase B is added to A and homogenized for 1 hour. Pre-mixed phase C is added. Pre-mixed phase D is added and the mixture is homogenized for 30 minutes while maintaining the temperature at 75-80° C. Phase E is added under the same conditions.

In a separate vessel phase F is melted until clear and uniform. Phase F is added to the mixture using a homogenizer for 15 minutes. The mixture is cooled to 40-45° C. Phase G and H are then added. The mixture is then cooled to room temperature.

This results a liquid O/W makeup foundation with good overall properties.

What is claimed is:

1. Microparticles containing an effective amount of at least one fluorescent whitening agent, wherein said fluorescent whitening agent is entrapped in a transparent or translucent matrix polymer that has been formed from a blend of monomers comprising a first monomer which is an ethylenically unsaturated ionic monomer and a second monomer which is an ethylenically unsaturated hydrophobic monomer which is capable of forming a homopolymer of glass transition temperature in excess of 50° C., wherein secondary particles are distributed throughout the matrix, which secondary particles comprise a hydrophobic polymer that has been formed from an ethylenically unsaturated hydrophobic monomer which is capable of forming a homopolymer having a glass transition temperature in excess of 50° C. and optionally other monomers, which hydrophobic polymer is different from that of the matrix polymer.

2. Microparticles according to claim 1, which have a particle size of between 1 and 60 microns.

3. Microparticles according to claim 1, wherein the hydrophobic polymer that is different from that of the matrix polymer is a polymeric amphipathic stabilizer.

4. Microparticles according to claim 1, wherein the matrix polymer is cross-linked.

5. Microparticles according to claim 1, wherein the fluorescent whitening agent is selected from the group consisting of 4,4′-bis-(triazinylamino)-stilbene-2,2′-disulfonic acids, 4,4′-bis-(triazol-2-yl)stilbene-2,2′-disulfonic acids, 4,4′-dibenzofuranyl-biphenyls, 4,4′-(diphenyl)-stilbenes, 4,4′-distyryl-biphenyls, 4-phenyl-4′-benzoxazolyl-stilbenes, stilbenyl-naphthotriazoles, 4-styryl-stilbenes, bis-(benzoxazol-2-yl) derivatives, bis-(benzimidazol-2-yl) derivatives, coumarins, pyrazolines, naphthalimides, triazinyl-pyrenes, 2-styryl-benzoxazole or -naphthoxazoles, benzimidazole-benzofurans and oxanilides, and mixtures thereof of fluorescent whitening agents of the same or different chemical classes.

6. Microparticles according to claim 1, wherein the fluorescent whitening agent is selected from the group consisting of 4,4′-bis-(triazinylamino)-stilbene-2,2′-disulfonic acids and 4,4′-dibenzofuranyl-biphenyls.

7. Microparticles according to claim 6, wherein the fluorescent whitening agent is a 4,4′-distyryl-biphenyls of the formula: 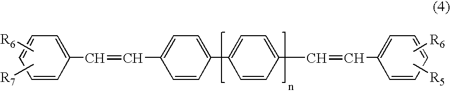
in which R₅ and R₆, independently, are H, SO₃M, SO₂N(C₁-C₄alkyl)₂, O—(C₁-C₄alkyl), CN, Cl, COO(C₁-C₄alkyl), CON(C₁-C₄alkyl)₂ or O(CH₂)₃N^{⊕}(CH₃)₂An⁻, in which M is H, Na, Li, K, Ca, Mg, ammonium, or ammonium that is mono-, di-, tri- or tetra-substituted by C₁-C₄alkyl, C₁-C₄-hydroxyalkyl or a mixture thereof, An⁻ is an anion of an organic or inorganic acid or a mixture thereof, and n is 1.

8. Microparticles according to claim 7, wherein the fluorescent whitening agent is a 4,4′-distyryl-biphenyls of the formula (4a)

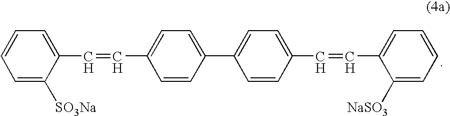

9. A solid or liquid personal care or cosmetic composition that comprises shatter-resistant microparticles as defined in claim 1 and a cosmetically tolerable carrier or adjuvant which is other than, or in addition to water.

10. A solid or liquid personal care or cosmetic composition according to claim 9, which comprises from 0.0001 to 10% by weight of the shatter-resistant microparticles

11. A composition according to claim 9, which is formulated as a water-in-oil or oil-in-water emulsion, as an alcoholic or alcohol-containing formulation, as a vesicular dispersion of an ionic or non-ionic amphiphilic lipid, as a gel, or a solid stick.

12. A composition according to claim 9, wherein the personal care or cosmetic composition is in the form of a shaving preparation, a skin-care preparation, a cosmetic personal care preparation or a light-protective preparation.

13. A composition according to claim 9, wherein the shaving preparation is an aftershave lotion or aftershave cream, the skin-care preparation is a skin emulsion, a multi-emulsion, a skin oil or a body powder, the cosmetic personal care preparation is a facial make-up in the form of lipstick, a day cream, a facial lotion or cream, and the light-protective preparation is a sun tan lotion, cream or oil, a sun block or a pretanning preparation.

14. A composition according to claim 9, which further comprises at least one further constituent selected from the group consisting of sequestering agents, non-encapsulated or encapsulated colorants, perfumes, thickening or solidifying (consistency regulator) agents, emollients, non-encapsulated or encapsulated UV absorbers, skin-protective agents, antioxidants and preservatives.

15. A method of masking or reducing the appearance of skin imperfections, which comprises applying a solid or liquid personal care or cosmetic formulation having an effective amount of shatter-resistant polymeric microparticles according to claim 1 to the surface of the skin.

16. A method according to claim 15, wherein the personal care or cosmetic composition comprises a blend of polymeric microparticles containing different microencapsulated fluorescent whitening agents that are individually provided in separate polymeric matrix materials.

17. A method according to claim 15, wherein the personal care or cosmetic composition comprises polymeric microparticles containing a blend of at least two different fluorescent whitening agents that are embedded in a single polymeric matrix material.

18. A method according to claim 15, wherein the personal care or cosmetic composition is in the form of a skin-care preparation, a cosmetic personal care preparation or a light-protective preparation.
